(19) United States
(12) Patent Application Publication
(10) Pub. No.: US 2007/0161994 A1
(43) Pub. Date: Jul. 12, 2007

(54) Hinged Polyaxial Screw and methods of use

(76) Inventors: Gary L. Lowery, Jacksonville, FL (US); Frank T. Trautwein, Filderstadt (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/537,070

(22) Filed: Sep. 29, 2006

Related U.S. Application Data
(60) Provisional application No. 60/722,337, filed on Sep. 30, 2005.

(51) Int. Cl. A61F 2/30

(52) U.S. Cl. 606/61

(57) ABSTRACT

A bone-anchoring device is provided. The device may include a substantially rigid shaft having a threaded portion configured to engage bone. The bone-anchoring device may further include a head portion securely attached to the shaft and a cap portion having a hinged connection with the head portion. A substantially spherical cavity may be formed between the head portion and the cap portion.

### FIELD OF THE INVENTION

The present invention relates to devices and methods for anchoring surgical implants to bony tissue. Specifically, the present invention pertains to polyaxial screws, which may be configured for use with bone-stabilization devices such as implantable rod stabilization systems.

### BACKGROUND OF THE INVENTION

Diseases of the spine cause significant morbidity. These diseases include abnormalities of the vertebrae, the intervertebral discs, the facet joints, and connective tissue around the spine. These abnormalities can be caused by a number of factors, including mechanical injury or degenerative disc disease. Such abnormalities can cause instability to the spine, vertebral misalignment, and abnormal motion between adjacent vertebrae. More severe disease may result in wear to the vertebral bony surfaces or cause nerve compression, which may ultimately produce severe pain. Further, spinal conditions are often chronic and progressive problems.

The treatments for spinal disorders may include long-term medical management or surgery. Medical management is generally directed at controlling the symptoms, such as pain, rather than correcting the underlying problem. For some patients this may require chronic use of pain medications, which may alter patient mental state or cause other negative side effects.

Another treatment option is surgery, which is often highly invasive and may significantly alter the spinal anatomy and function. For example, one surgical treatment for certain spinal conditions includes spinal fusion, whereby two or more vertebrae may be joined using bone grafts and/or synthetic implants. Fusion is irreversible and may significantly alter vertebral range-of-motion. Further, current surgical procedures are often only applicable to patients in a significantly progressed disease state.

Consequently, spinal surgeons have begun to develop more advanced surgical procedures and spinal stabilization and/or repair devices that are less invasive, may be reversible, and cause a less drastic alteration in the patient's normal anatomy and spinal function. These procedures may be used in an earlier stage of disease progression and, in some situations, may even stop or reverse disease progression.

For some surgical procedures and stabilization implants, it is desirable to use a bone-anchoring element that can be implanted in a variety of configurations. For example, it is often desirable to use bone screws that can be fixed to bone at a range of suitable angles and still be properly connected with other components of an integrated treatment system.

Recently, spinal surgeons have begun to develop more dynamic treatment systems. Such systems may provide a certain degree of limited but controlled movement and may provide improved care for patients suffering from a variety of disorders including, for example, scoliosis and degenerative disc disease. These systems may benefit from improved bone-anchoring elements, including polyaxial screws.

### SUMMARY OF THE INVENTION

One aspect of the present invention includes a bone-anchoring device. The device may include a substantially rigid shaft having a threaded portion configured to engage bone. The bone-anchoring device may further include a head portion securely attached to the shaft and a cap portion having a hinged connection with the head portion. A substantially spherical cavity may be formed between the head portion and the cap portion.

A second aspect of the present invention includes a bone-anchoring system. The device may include an anchor having a substantially rigid shaft and further having a threaded portion configured to engage bone. The anchor may also include a head portion securely attached to the shaft and a cap portion having a hinged connection with the head portion. A substantially spherical cavity may be formed between the head portion and the cap portion. The system may further include a substantially spherical member configured to engage a rod and to be securely disposed within the substantially spherical cavity.

It is to be understood that both the foregoing general description and the following detailed description are exemplary and explanatory only, and are not restrictive of the invention, as claimed.

The accompanying drawings, which are incorporated in and constitute a part of this specification, illustrate several embodiments of the invention and together with the description, serve to explain the principles of the invention.

Additional objects and advantages of the invention will be set forth in part in the description which follows or may be learned by practice of the invention. The objects and advantages of the invention will be realized and attained by means of the elements and combinations particularly pointed out in the appended claims.

### BRIEF DESCRIPTION OF THE DRAWINGS

FIG. 1A illustrates an exploded view of a bone-anchoring device and rod-connection system, according to an exemplary disclosed embodiment.

FIG. 1B illustrates a perspective view of a rod connector according to an exemplary disclosed embodiment.

FIG. 1C illustrates a perspective view of a rod connector according to an exemplary disclosed embodiment.

FIG. 2A illustrates a cross-sectional view of an assembled bone-anchoring device and rod-connection system, according to an exemplary disclosed embodiment.

FIG. 2B illustrates a front-to-back view of an assembled bone-anchoring device and rod-connection system, according to an exemplary disclosed embodiment.

FIG. 2C illustrates a perspective view of an assembled bone-anchoring device and rod-connection system, according to an exemplary disclosed embodiment.

### DESCRIPTION OF THE PREFERRED EMBODIMENTS

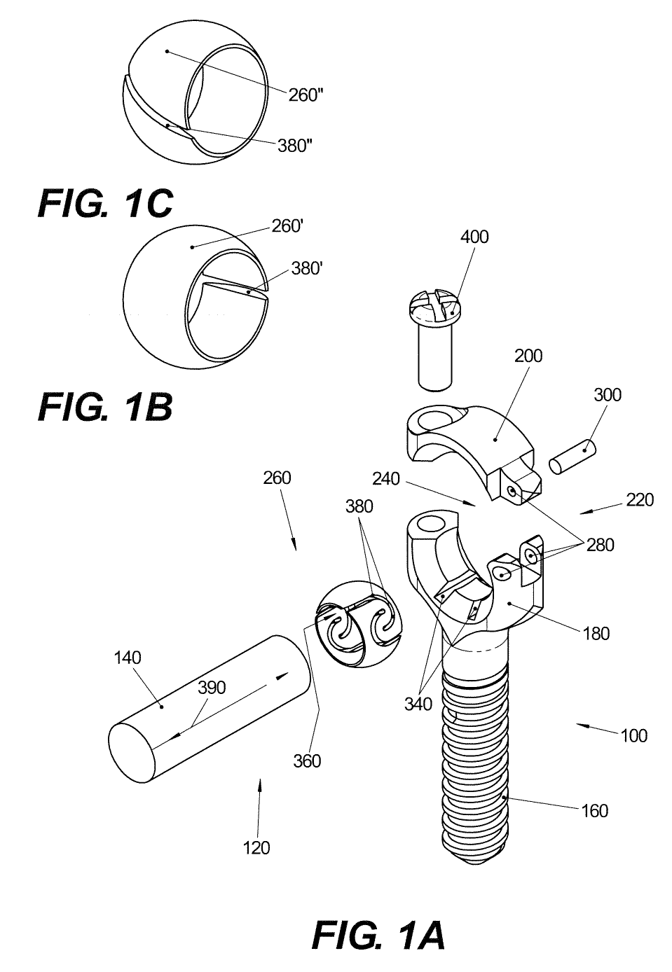

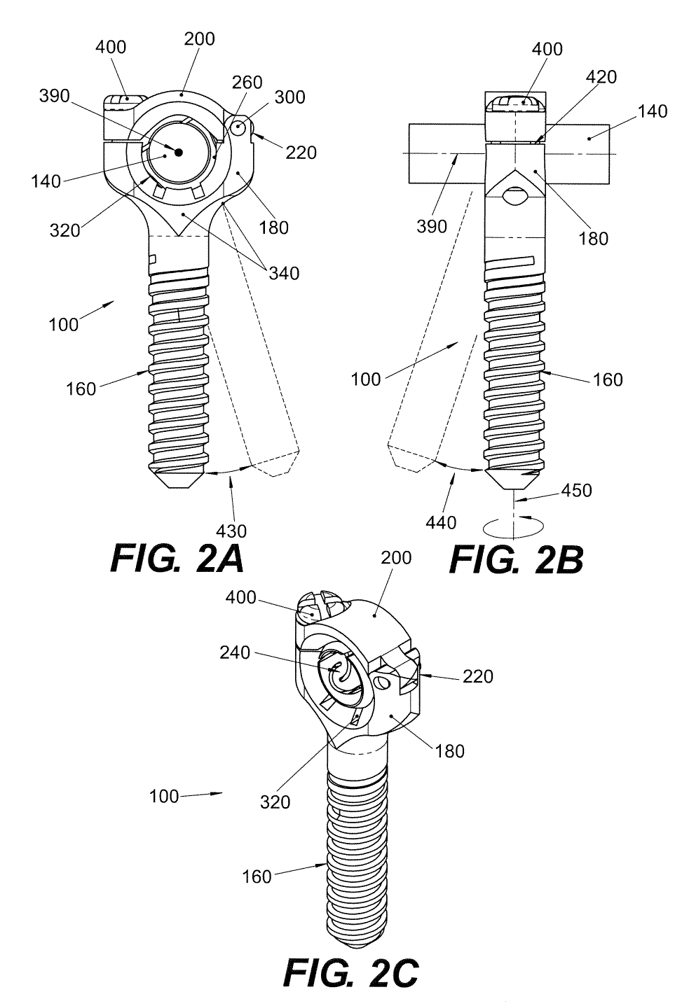

FIG. 1A illustrates the component parts of a bone-anchoring device 100 and rod-connection system 120, according to an exemplary embodiment. The bone-anchoring device 100 may include a threaded shaft 160, which may be configured to securely engage one or more bony structures. The bone-anchoring device 100 may further include a head portion 180 securely attached to the shaft 160 and a cap 200. The cap 200 may form a hinged connection 220 with the head portion 180, and collectively, the head portion 180 and the cap 200 may form part of a substantially spherical cavity 240. A substantially spherical connector 260 may be provided as part of the rod-connection system 120 to facilitate secure connection of the bone-anchoring device 100 and an implant such as a stabilization rod 140, as shown in FIGS. 2A and 2B. The connector 260 may be configured to be disposed within the cavity 240 during use.

As shown, the stabilization rod 140 comprises a cylindrical rod. However, it is understood that the rod 140 may comprise any type or kind of implantable rod suitable for surgical application to a patient. In an exemplary application, such rods may be implanted at one or more locations along the vertebral column to facilitate alignment and/or stabilization of the spine. Further, in some cases, suitable stabilization rods may be used with or without other treatments to correct spinal deformities, such as scoliosis. Additionally, rods may provide stabilization to treat diseases of the discs, facet joints, ligaments, and/or any other anatomical structure that may affect the spine.

In addition, the stabilization rod 140 may cooperate with one or more additional components to form an implantable treatment system. For example, in one embodiment, the stabilization rod 140 may be secured to one or more bones, including one or more vertebrae, a sacrum, or any other suitable bony structure. Further, the stabilization rod 140 may form a flexible or rigid connection with additional implantable components, including for example, interspinous stabilization systems, dynamic posterior stabilization devices, laminar or pedicle hooks, vertebral body prostheses, vertebral disc prostheses, and/or any other suitable implantable device.

The shaft 160 of the bone-anchoring device 100 may include a number of suitable configurations. For example, the shaft 160 may include a variety of suitable shapes, lengths, materials, and/or physical properties. The specific shape, size, and/or materials of the shaft may be selected based on the desired implant location, the physical and/or biological conditions to which the device may be exposed, and whether the device will be permanently or temporarily implanted.

In one embodiment, the shaft 160 may include a threaded portion, which may be configured to securely engage one or more bony structures. The specific thread design may be selected from numerous suitable designs. For example, many suitable thread designs are available for various bone screws. The appropriate thread design may be selected based on the targeted anatomical location, general bone health, and/or projected length of use. In addition, suitable thread designs can have a variety of different cross-sectional shapes, such as for example, polygonal, circular, or quadratic shapes. Further, the screw threads may be of uniform depth along the screw length, or the thread depth may vary along the screw length. For example, in one exemplary embodiment, the screw may have a thread depth that decrease towards the head portion 180, as shown in FIGS. 1A and 2A-2C.

The bone-anchoring device 100 may be produced from a variety of suitable materials. Furthermore, each of the components of the bone-anchoring device 100 may be produced from a single material. Alternatively, the bone-anchoring device 100 may be produced from multiple different materials. For example, in one embodiment, the shaft 160, which may be implanted into a bony structure, may be produced from a material having certain physical properties, as well as suitable biocompatibility. Other components, such as portions of the head 180 or cap 200, may be produced from materials having very durable physical properties, which may ensure a reliable and permanent connection with the stabilization rod 140.

In one embodiment, the bone-anchoring device 100 may include a biocompatible material. For example, the bone-anchoring device 100 may include a number of suitable biocompatible metals, ceramics, composites, and/or polymeric materials. Such materials may include, for example, titanium, stainless steel, cobalt chrome, zirconia, nickel-titanium alloys, PEEK, polyethylene, and/or any other suitable material. The specific material may be selected based on desired physical properties including, for example, a desired modulus of elasticity, strength, fracture toughness, and/or any other suitable mechanical property.

The head portion 180 may be securely connected to the shaft 160. For example, in one embodiment, the head portion 180 and the shaft 160 may be constructed as a single component. Alternatively, the head portion 180 and shaft portion 160 may be fabricated individually and securely connected later in production. If fabricated as individual components, the head 180 and shaft 160 may be connected using any suitable process. For example, the materials that form the head 180 and shaft 160 may be welded by arc welding, laser welding, and/or any other suitable welding process. Alternatively or additionally, the shaft 160 and the head portion 180 may be securely engaged using, for example, a threaded connection, press-fit connection, or form-fit or snap-in connection.

As previously described, the cap 200 may form a hinged connection 220 with the head portion 180. Any suitable hinged connection may be used. For example, as shown in FIG. 1A, the head portion 180 and the shaft 160 may each include one or more hinge openings 280 through which a hinge connector 300 may be placed. In one embodiment, the hinge connector 300 may include, for example, a cylindrical rod or pin configured to form a press-fit connection with the hinge openings 280. Alternatively, the hinge connector 300 may include a threaded connector such as a screw, a bolt, a nut and bolt combination, or any other suitable connector.

Before implantation, the bone-anchoring device 100 may be disassembled, partially assembled, or completely assembled. For example, in one embodiment, the bone anchoring device 100 may be provided as separate components, and a surgeon may assemble the components prior to or during surgery. Particularly, a surgeon may be provided with the shaft 160 and the head portion 180, which the surgeon may securely fix to bone. The surgeon may then assemble the hinged connection 220 to connect the cap 200 to the head 180. Alternatively, the surgeon may be provided with the bone-anchoring device 100 having the cap 200, which is already secured to the head 180 by the hinged connection 220. In this way, the surgeon will not have to spend extra time and effort assembling the bone-anchoring device 100 and will not risk losing one or more small components or incorrectly assembling the bone-anchoring device 100.

The substantially spherical cavity 240 may be configured to securely receive the connector 260. Further, the connector 260 and the cavity 240 may be configured to form a releasable or permanent connection. For example, in one embodiment, the head portion 180 may be configured to form a snap-fit connection with the connector 260.

FIG. 2A shows a side view of the bone-anchoring device 100, including the connector 260 and the implant 140. In this embodiment, the head portion 180 is shown to have an edge 320, which forms an arc of at least, and preferably greater than, 180°. The arc, being greater than 180°, may produce a certain amount of pressure on the surface of the connector 260 during placement of the connector 260 within the cavity 240, producing a snap-fit connection.

In addition, the head 180 may be configured to have a certain amount of flexibility, to facilitate placement of the connector 260 within the cavity 240, using a snap-fit connection. For example, in one embodiment, the head portion 180 may be formed from a material having a certain degree of flexibility. Suitable materials may have a certain modulus of elasticity and may include certain metals, such as titanium. Alternatively or additionally, the head may include one or more notches 320 or grooves (as shown in both FIGS. 1A and 2A), which may provide thinner sections of the head 180. The notches 320 may facilitate lateral expansion of the cavity 240, thereby allowing placement of the connector 260 within the cavity 240.

The connector 260 may also be configured to compress or expand slightly. Compression and expansion of the connector 260 may serve several purposes. For example, in one embodiment, the connector 260 may be provided as a component that is separate from the stabilization rod 140, and compression and/or expansion of the connector 260 may facilitate secure placement of the connector 260 on the stabilization rod 140. In addition, compression of the connector 260 may facilitate placement of the connector 260 within the cavity 240, particularly when the cavity 240 and the connector 260 are configured to form a snug or snap-fit connection.

Compression and expansion of the connector 260 may be effected in a number of suitable ways. For example, in one embodiment, the connector may be produced from a material having a certain elastic modulus. Alternatively or additionally, the connector 260 may include one or more structural features that may provide compression or expansion. For example, as shown in FIG. 1A, the connector 260 may include one or more surface gaps 380 or notches. The gaps 380 may allow the connector 260 to compress or expand. Such compression or expansion of the gaps 380 will narrow or widen an opening 360 in the connector 260, through which the stabilization rod 140 may be passed.

The connector 260 and the stabilization rod 140 may be provided in a number of suitable configurations. For example, in one embodiment, the connector 260 and the stabilization rod 140 may be provided as separate components, and a surgeon may assemble the components by placing the stabilization rod 140 within the opening 360 of the connector. Alternatively, the connector 260 and implant may be preassembled.

The connector 260 may be provided in a number of suitable configurations. For example, as shown, the connector 260 includes a ring with a rounded outer surface. The rounded outer surface provides a substantially spherical shape, which will fit within the cavity 240. In addition, the ring-shaped connector 260 may include surface gaps 380, which provide compressibility and/or expandability to the ring. Further, as shown, the surface gaps 380 can include opposed S-shaped gaps 380 or notches. However, any suitable gap shape or configuration may be used. For example, the gaps 380 may include one gap 380, two gaps 380, three gaps 380, or any other suitable number of gaps 380. In addition, gaps 380 may include S-shaped gaps 380 (as shown in FIG. 1A), linear gaps, or any other suitable configuration. For example, in one embodiment, as shown in FIG. 1B, a connector 260′ includes a linear gap 380′ directed straight across the width of the connector 260′. In another embodiment, as shown in FIG. 1C, a connector 260″ includes a linear gap 380″ directed at an angle across the width of the connector 260″.

In addition, the connector 260 and the stabilization rod 140 may be connected in a number of suitable manners. For example, in one embodiment, the connector 260 may be rigidly fixed to the stabilization rod 140 or constructed as one piece. In another embodiment, the connector 260 may be configured to slide along a longitudinal axis 390 of the stabilization rod 140 before or after implantation. In still another embodiment, the connector 260 may rotate around the longitudinal axis 390 of the stabilization rod 140. Further, the connector 260 may rotate in the cavity 240 after closure of the cap 200. Rotation of the connector 260 may allow the rod 140 to adapt in relative angular position, which may be desirable in a dynamic treatment system.

During use, a surgeon may select a preassembled stabilization rod 140 and connector 260, or may connect the stabilization rod 140 and the connector 260 in a desired configuration. The surgeon may then place the connector 260 within the substantially spherical cavity 240 of a bone-anchoring device 100 that has been properly secured to a bony tissue. Further, the snap-fit configuration may allow a surgeon to position the connector 260 within the cavity 240 and to remove and reposition one or more components as the surgery progresses.

After the surgeon has properly positioned the connector 260 and the stabilization rod 140, the surgeon may position the cap 200 over the connector 260 to secure the connector 260 within the cavity 240. The cap 200 may rotate with respect to the hinge connector 300, thereby allowing the cavity 240 to be opened or closed. The cap 200 and the head portion 180 may be configured to receive a locking device 400. The locking device 400 will allow a surgeon to fix the cap 200 in a closed position with respect the head 180 and hinged connection 220. In one embodiment, the locking device 400 is disposed opposite the hinged connection 220 with respect to the cavity 240. In another embodiment, the locking device 400 is disposed on the same side of the cavity 240 on which the hinged connection 220 is located.

The locking device 400 may include a number of suitable locking devices. For example, the locking device 400 may include a threaded device, such as a screw, a bolt, or a nut and bolt combination. The locking device 400 may also include a press-fit connector. Any suitable locking device 400 may be selected.

The bone-anchoring device 100 may be configured to provide the surgeon with some choice as to how tightly to close the cap 200. As shown in FIG. 2B, the cap 200 and the head portion 180 may include a gap 420 where the locking device 400 is located. In some embodiments, a surgeon may tighten or loosen the locking device 400 to increase or decrease the size of the gap 420.

Controlling the size of the gap 420 may allow a certain degree of movement of the bone-anchoring device 100 with respect to the stabilization rod 140. For example, in one embodiment, the surgeon may produce a tight connection between the connector 260 and the cavity 240 by tightening the locking device 400. The tight connection may prevent any rotational movement of the bone-anchoring device 100 about the connector 260. Alternatively, the surgeon can select a configuration that allows the bone-anchoring device 100 to rotate freely or with a certain degree of resistance. The specific degree of movement may be selected based on the desired clinical application and patient characteristics. It should be noted that the surgeon may select desired degrees of movement, resistance, or any other implant feature by controlling how the device is implanted, how the components are assembled, and/or by selecting implants designed to provide desired features.

The bone-anchoring device 100, having the substantially spherical cavity 240, may engage the connector 260 at a range of suitable angles and, as noted above, may maintain a certain degree of rotational mobility with respect to the connector 260. The variable engagement and rotational mobility of the implant may facilitate implantation of the bone-anchoring device 100 and the stabilization rod 140, while also producing desired clinical outcomes. For example, the ability to rotate the bone-anchoring device 100 with respect to the stabilization rod 140 would allow the surgeon to connect the bone-anchoring device 100 at a range of angles, thereby providing more flexibility during surgery. In addition, after implantation, the bone-anchoring device 100 may maintain some degree of mobility with respect to the stabilization rod 140. This continued mobility after implantation may facilitate connection of some dynamic treatment systems, which may be configured to provide controlled but sustained movement of the spine.

In the present embodiment, the bone-anchoring device 100 coupled with the connector 260 enables rotation of the stabilization rod 140 in three degrees of freedom with respect to the bone anchoring device 100. As noted, the spherical connector 260 may be configured to rotate within the substantially spherical cavity 240, thereby allowing rotation of a rod 140 connected to the spherical connector 260. As shown in FIG. 2C, the spherical connector 260 and rod 140 can be configured to rotate about any or all of three X, Y, and Z axes along directions A, B, and C, respectively. In some embodiments, the connector 260 and rod 140 can be configured to rotate up to 360° about the axis 390 of the rod 140. Further, the connector 260 and the rod 140 may be configured to rotate a certain amount with respect to both the X and Z axes. For example, the connector and the rod 140 may be configured to rotate within a range of about −45° to about 45°, about −30° to about 30°, or about −15° to about 15°, about either or both of the X and Z axes. The specific amount of rotation may be controlled by selecting an appropriately sized connector 260, cavity 240, and/or rod 140. Further, as noted previously, the connector 260 may be rigidly fixed to the rod 140 or may rotate or slide with respect to the rod 140.

The cavity 240 and/or the connector 260 may also include one or more surface lining materials. Such materials may include a variety of suitable surface-lining materials. These materials may be selected based on desired physical properties including, for example, certain tribologic properties or the ability to absorb impact. For example, in one embodiment, the cavity 240 may be lined with a material having a low friction coefficient with respect to the surface of the connector 260. In one embodiment, the cavity 240 may have a surface including a polyethylene material, such as for example, ultra high molecular weight polyethylene (UHMWPE).

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A bone-anchoring device, comprising:
a substantially rigid shaft having a threaded portion configured to engage bone;
a head portion securely connected to the shaft;
a cap portion having a hinged connection with the head portion; and
a substantially spherical cavity formed between the head portion and the cap portion.

2. The device of claim 1, further including a fastening device for securing the cap to the head.

3. The device of claim 2, wherein the hinged connection is located on a first side of the cap with respect to the spherical cavity, and the fastening device is located on a second side of the cap with respect to the spherical cavity.

4. The device of claim 2, wherein the hinged connection and the fastening device are located on the same side of the cap with respect to the spherical cavity.

5. The device of claim 2, wherein the fastening device includes a screw.

6. The device of claim 2, wherein the fastening device includes a bolt.

7. The device of claim 2, wherein the fastening device includes a press-fit fastener.

8. The device of claim 1, wherein the hinged connection includes a hinge fastener.

9. The device of claim 8, wherein the hinge fastener includes a screw.

10. The device of claim 8, wherein the hinge fastener includes a bolt.

11. The device of claim 8, wherein the hinge fastener includes a press-fit fastener.

12. The device of claim 1, wherein the head portion includes a lower edge forming greater than 180 degrees of the substantially spherical cavity.

13. The device of claim 1, wherein the head portion includes a lower edge forming less than or equal to 180 degrees of the substantially spherical cavity.

14. The device of claim 1, wherein the head portion is configured to form a snap-fit connection with a substantially spherical member.

15. The device of claim 14, wherein the head portion includes one or more grooves.

16. The device of claim 14, wherein the head portion is formed of a material selected from the group including titanium, a steel, cobalt-chrome, stainless steel, and a polymeric material.

17. A bone-anchoring system, comprising:
an anchor, including:
a substantially rigid shaft having a threaded portion configured to engage bone;
a head portion securely connected to the shaft;
a cap portion having a hinged connection with the head portion; and
a substantially spherical cavity formed between the head portion and the cap portion; and
a substantially spherical member configured to engage a rod and to be securely disposed within the substantially spherical cavity.

18. The system of claim 17, further including a fastening device for securing the cap to the head.

19. The system of claim 18, wherein the hinged connection is located on a first side of the cap with respect to the spherical cavity, and the fastening device is located on a second side of the cap with respect to the spherical cavity.

20. The system of claim 18, wherein the hinged connection and fastening device are located on the same side of the cap with respect to the spherical cavity.

21. The system of claim 18, wherein the fastening device includes a screw.

22. The system of claim 18, wherein the fastening device includes a bolt.

23. The system of claim 18, wherein the fastening device includes a press-fit fastener.

24. The system of claim 17, wherein the hinged connection includes a hinge fastener.

25. The system of claim 24, wherein the hinge fastener includes a screw.

26. The system of claim 24, wherein the hinge fastener includes a bolt.

27. The system of claim 24, wherein the hinge fastener includes a press-fit fastener.

28. The system of claim 17, wherein the head portion includes a lower edge forming greater than 180 degrees of the substantially spherical cavity.

29. The system of claim 17, wherein the head portion includes a lower edge forming less than or equal to 180 degrees of the substantially spherical cavity.

30. The system of claim 17, wherein the head portion is configured to form a snap-fit connection with the substantially spherical member.

31. The system of claim 30, wherein the head portion includes one or more grooves.

32. The system of claim 30, wherein the head portion is formed of titanium.

33. The system of claim 17, wherein the substantially spherical member is configured to removably engage a rod.

34. The system of claim 17, wherein the substantially spherical member is configured to slidingly engage a rod.

35. The system of claim 17, wherein the substantially spherical member is configured to rigidly engage a rod.

36. The system of claim 17, wherein the substantially spherical member is configured to permanently engage a rod.

37. The system of claim 17, wherein the substantially spherical member rotates within the substantially spherical cavity of the anchor.

38. The system of claim 17, wherein the anchor is configured to be connected to the substantially spherical member at a range of angles with respect to the rod.

39. The system of claim 17, wherein the substantially spherical member is compressible.

40. The system of claim 17 wherein the substantially spherical member comprises a ring.

41. The system of claim 17, wherein the substantially spherical member includes at least one notch.

42. The system of claim 17, wherein the substantially spherical member includes at least one surface gap.

43. The system of claim 17, wherein the rod is configured to treat a curvature of the spine.

44. The system of claim 17, wherein the rod is configured to facilitate spinal fusion.

45. The system of claim 17, wherein the rod is configured to secure an implantable treatment system.
